(19) United States
(12) Patent Application Publication
(10) Pub. No.: US 2025/0134447 A1
(43) Pub. Date: May 1, 2025

(54) SIMULTANEOUS ASSESSEMENT OF AFFERENT AND EFFERENT VISUAL PATHWAYS

(71) Applicant: The Regents of the University of California, Oakland, CA (US)

(72) Inventors: Jennifer Graves, Encinitas, CA (US); Tzyy-Ping Jung, San Diego, CA (US); Masaki Nakanishi, San Diego, CA (US)

(21) Appl. No.: 18/690,237

(22) PCT Filed: Sep. 12, 2022
(86) PCT No.: PCT/US2022/076273

§ 371 Date: Mar. 7, 2024

Related U.S. Application Data
(60) Provisional application No. 63/243,098, filed on Sep. 11, 2021.

(51) Int. Cl. A61B 5/00; A61B 3/113; A61B 5/378; A61B 5/398

(52) U.S. Cl. CPC A61B 5/4076; A61B 3/113; A61B 5/378; A61B 5/398; A61B 5/4047; A61B 5/4052; A61B 5/742

(57) ABSTRACT

Methods and systems for assessing afferent and efferent visual functions are disclosed. In one aspect, a wearable system for concurrently assessing afferent and efferent visual functions includes a display configured to be placed in front of a face of a user and provide visual stimuli to the user to elicit eye movements, an electroencephalography (EEG) sensor configured to be placed on a head of the user to measure electrical activity in a brain of the user that occurs in response to the visual stimuli, an eye-tracker configured to track the eye movements, and a processor coupled to the display, the electroencephalography sensor, and the eye-tracker to: cause the visual stimuli to be presented on the display; obtain an electroencephalography signal from the EEG sensor; obtain eye-tracking measurements from the eye-tracker; and determine, based on the electroencephalography signal and the eye-tracking measurements, information associated with the afferent and efferent visual functions.

### CROSS-REFERENCE TO RELATED APPLICATION

This patent document claims priority to and benefits of U.S. Provisional Appl. No. 63/243,098, entitled “SIMULTANEOUS ASSESSMENT OF AFFERENT AND EFFERENT VISUAL PATHWAYS USING MULTI-FOCAL STEADY-STATE VISUAL EVOKED POTENTIAL METHOD TO FACILITATE THE DIAGNOSIS AND PROGNOSIS OF INDIVIDUALS WITH NEUROLOGICAL DISEASES” and filed on Sep. 11, 2021. The entire contents of the before-mentioned patent application are incorporated by reference as part of the disclosure of this document.

### TECHNICAL FIELD

This patent document relates to systems, devices, and processes that use brain computer interface technologies.

### BACKGROUND

Multiple sclerosis (MS) is a leading cause of neurological disability. An autoimmune disorder, MS targets the optic nerves, brain and spinal cord, primarily damaging the myelin sheaths of neurons in these structures with secondary neurodegeneration. Both the afferent and efferent visual pathways are strongly impacted by MS pathology. Over 50% of MS patients will present with acute optic neuritis (ON) during their disease course, with 20% having ON as their first symptom. Up to 70% of patients also experience efferent visual dysfunction, including ocular misalignment, nystagmus and abnormal eye-tracking. Both afferent and efferent functions can be precisely measured to aid diagnosis, estimate treatment responses, and follow the course of neurodegeneration/disability accumulation. Unfortunately, the equipment and analytical capacity to make these measurements are typically only found in specialty tertiary care facilities, and even then, few centers can accurately quantify both afferent and efferent dysfunction. There is no current availability to make these measurements in acute care centers such as emergency room (ER), urgent care, and hospital floors.

### SUMMARY

Disclosed are methods, systems, and devices for concurrently assessing afferent and efferent visual functions.

In an implementation of the disclosed technology, a wearable system for assessing afferent and efferent visual functions includes a display configured to be placed in front of a face of a user and provide visual stimuli to the user to elicit eye movements, an electroencephalography (EEG) sensor configured to be placed on a head of the user to measure electrical activity in a brain of the user that occurs in response to the visual stimuli, an eye-tracker configured to track the eye movements, and a processor coupled to the display, the electroencephalography sensor, and the eye-tracker to: cause the visual stimuli to be presented on the display; obtain an electroencephalography signal from the EEG sensor; obtain eye-tracking measurements from the eye-tracker; and determine, based on the electroencephalography signal and the eye-tracking measurements, information associated with the afferent and efferent visual functions.

In another implementation of the disclosed technology, a method of quantifying multiple sclerosis related visual dysfunctions includes displaying a series of images to elicit eye movements of a user, obtaining visual-evoked potential data and eye-tracking data from the user while displaying the series of images, and determining information associated with afferent signal conduction and efferent oculomotor function based on the visual-evoked potential data and the eye-tracking data.

Those and other features are described in greater detail in the drawings, the description and the claims.

### BRIEF DESCRIPTION OF THE DRAWINGS

FIG. 1A shows a central nervous system. FIG. 1B shows two visual systems.

FIG. 2A shows a system that is used for an experiment according to an example embodiment. FIG. 2B shows another view of the system shown in FIG. 2A to illustrate a virtual reality (VR) head-mounted display (HMD) part of the system placed on top of an EEG cap. FIG. 2C shows an example sequence of movements of a visual flicker based on an example embodiment of the disclosed technology.

FIG. 3A shows example amplitude spectra of multi-focal steady-state visual evoked potential (mfSSVEPs). FIG. 3B shows example electromyography (EOG)-based eye tracking data for a control subject and a multiple sclerosis (MS) patient.

FIG. 4 shows the averaged mfSSVEP amplitude and the performance of pursuit task in the groups of healthy controls and MS patients.

FIG. 5 shows a device implemented based on an example embodiment.

FIG. 6 shows some example results of visual field assessments from nGoggle mfSSVEP and standard perimetry of glaucomatous and healthy eyes.

FIG. 7 shows a signal processing pipeline that can be used to quantify mfSSVEP signals based on some embodiments of the disclosed technology.

FIG. 8 shows example traces of angular position in a smooth pursuit experiment.

FIG. 9 shows an example of a wearable system for assessing afferent and efferent visual functions based on some embodiments of the disclosed technology.

FIG. 10 shows an example method of quantifying multiple sclerosis related visual dysfunctions based on some embodiments of the disclosed technology.

### DETAILED DESCRIPTION

Disclosed in this patent document are novel mobile brain-computer interfacing methods and systems for concurrently assessing afferent and efferent visual functions. Among other benefits, the disclosed methods and systems can facilitate research on the correlations between the afferent and efferent measures and support the development of new and novel indices of multiple sclerosis (MS)-related visual dysfunction.

As discussed above, both afferent and efferent functions can be precisely measured to aid diagnosis, estimate treatment responses, and follow the course of neurodegeneration/disability accumulation. There is no current availability to make these measurements in acute care centers (ER, urgent care, hospital floors). The disclosed technology can be implemented in some embodiments to address these gaps via the application of an integrated wearable device capable of capturing both high-resolution afferent function and eye-tracking measurements in the MS population.

One of the greatest unmet needs in multiple sclerosis research and clinical care is the ability to quantify disease burden with highly reproducible and rater-independent methods. Most clinical trials and routine management decisions are based on a physical exam created over a century ago with tools invented in the 1880s (e.g., reflex hammer, tuning fork). The workhorse of disability metrics in MS, the expanded disability status scale (EDSS), is highly rater-dependent. It is based on the 150-year-old exam and poorly captures the mild to moderate visual dysfunction that is highly prevalent in the MS population. Significant advancements have been made in measuring afferent visual dysfunction through the use of low-contrast letter acuity measures, visual evoked potentials (VEP), optical coherence tomography (OCT), and oculometrics, but these tools are not in widespread use in most MS clinics and often require support from those with subspecialty training in neuro-ophthalmology. Most of the equipment is cumbersome and immovable. The functional vision measures also typically require performing a correction of refractive error, which most neurologists are not equipped to provide in their offices.

Despite the inaccessibility and current low-frequency use of extensive, systematic visual testing in neurology clinics or acute care settings, the visual system has demonstrated exquisite ability to detect demyelinating injury even in those without clinical symptoms. Standard full-field VEP has been used to support the diagnosis of MS for over 50 years. In young patients with MS, without any detectable exam finding abnormalities (e.g., EDSS score=0), latency delays can be seen for saccadic (fast) eye movements that distinguish these patients from healthy controls. The current understanding of the quantitative relationships between structure and function in the visual system far surpasses that of other central nervous system pathways. Thus, the afferent and efferent visual pathways are well poised to serve as a model system of injury and repair for therapeutic investigations and clinical monitoring in multiple sclerosis.

Early misdiagnosis remains a critical issue in MS, particularly for those with visual symptoms, and leads to delays in appropriate treatment and missed opportunities to prevent damage from the next attack. Many patients report months to years of struggling to get the correct diagnosis. One of the struggles for those with acute episodes of MS-related optic neuritis is that on exam the eye “looks” normal. This can lead to the ON diagnosis being missed. The majority of MS patients are not diagnosed or cared for in clinic settings with advanced visual function testing facilities. A mobile platform that does not require dedicated room space or a trained technician, such as the one according to the technology disclosed herein, would allow for more widespread application of quantitative afferent/efferent vision testing in emergency rooms and non-tertiary care centers.

Efferent visual function testing is currently only used in observational research settings. Despite the proven ability to detect dysfunction, quantification of dysfunction has not been brought to the clinic or clinical trial setting. This situation is largely due to the lack of technical expertise and immovable equipment needed to perform these measurements. As an example, one of the most commonly used platforms for MS studies requires each user to write their own software, which is not realistic for clinical practice neurologists.

Systems and methods disclosed herein will address these gaps and needs through providing a mobile assessment of simultaneously measured afferent and efferent visual dysfunction. According to some example embodiments, a portable, objective multi-focal steady-state visual evoked potential (mfSSVEP)-based brain-computer interface (BCI) platform with an embedded eye-tracking component can be used to quantify MS-related visual dysfunction in any setting (clinical, research or home).

A wearable (and/or wireless) mfSSVEP platform according to the disclosed technology can be used to detect demyelination-related afferent visual pathway injury in the eyes of MS patients. mfSSVEP signals captured by the platform can be used to differentiate diseased from healthy eyes. Such a platform can be used to quantitatively assess neural markers of demyelination-related afferent visual pathway injury.

Systems and methods according to the disclosed technology can employ concurrently collected eye-tracker signals (e.g., the eye-tracker signals can be also collected concurrently with mfSSVEP signals) to evaluate oculomotor function. Greater fixation losses and impaired smooth and saccadic eye movements can be expected in those with MS vs healthy controls and, furthermore, these abnormalities can be expected to track with the patient's overall disability level (expanded disability status scale score, EDSS). Therefore, data and information provided by devices according to the disclosed technology can be used as a biomarker of disease severity.

Systems and methods according to the disclosed technology can use metrics based on afferent and/or efferent measurements for detecting pathology. With an unprecedented ability to track these measures concurrently, the disclosed technology can also be implemented in some embodiments to take afferent and efferent visual function interdependence into account.

Systems according to some example embodiments include a mobile multi-focal steady-state VEP (mfSSVEP)-based brain-computer interface (BCI) platform with an embedded eye-tracking component that can simultaneously capture data on afferent and efferent visual function. The multi-focal stimulus design implemented in such systems would lead to faster assessment and better signal-to-noise ratio (SNR). A mobile platform that does not require dedicated room space or a trained technician would allow for more widespread application of quantitative afferent/efferent vision testing in acute care settings and non-tertiary care centers. Methods and systems according to the disclosed technology can allow removing the need for dedicated room space and for local technical and signal-processing expertise. The use of a headset platform according to some example embodiments can also allow the testing to be brought to the patient rather than having to burden the patient with a trip to an offsite research lab. Removing these critical barriers will, among other benefits, facilitate the use of efferent visual outcome measures in clinical trials and clinic settings.

An example embodiment of the portable BCI platform disclosed herein employs a unique, moving visual flicker (also referred to as an image or an icon) to capture both (a) delays in afferent signal conduction to the occipital cortex and (b) efferent oculomotor dysfunction. A user of the platform can fix their gaze at the center of the stimulus (e.g., the flicker) so that it would simultaneously elicit VEPs and saccadic eye movements. The amplitude of VEPs and saccadic eye movements are quantified by electroencephalography (EEG) and electromyography (EOG)/an eye-tracker (also referred to as eye tracker), respectively, to assess afferent and efferent visual dysfunction.

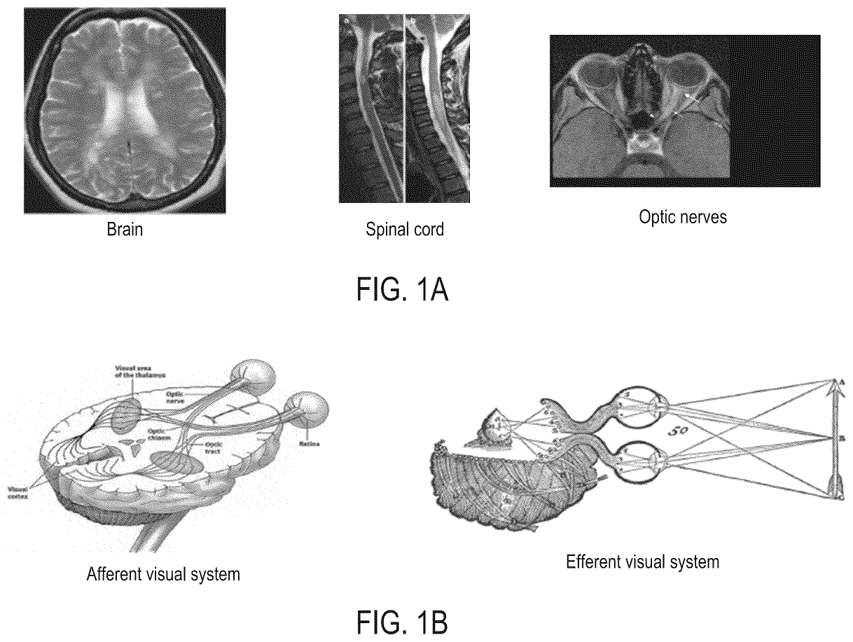

FIG. 1A shows a central nervous system. FIG. 1B shows two visual systems.

Referring to FIG. 1A, autoimmune disorder can be triggered by genetic and environmental factors and characterized by demyelination of the central nervous system (brain, spinal cord and optic nerves). Referring to FIG. 1B, visual systems include an afferent visual system relates to photon absorption to the occipital cortex, and an efferent visual system relates to a control of eye position and fixation.

Both the afferent and efferent visual pathways are strongly impacted by MS pathology. 50% of MS patients will present with acute optic neuritis (ON) during their disease course. Up to 70% of patients experience efferent visual dysfunction, including ocular misalignment, nystagmus and abnormal eye-tracking.

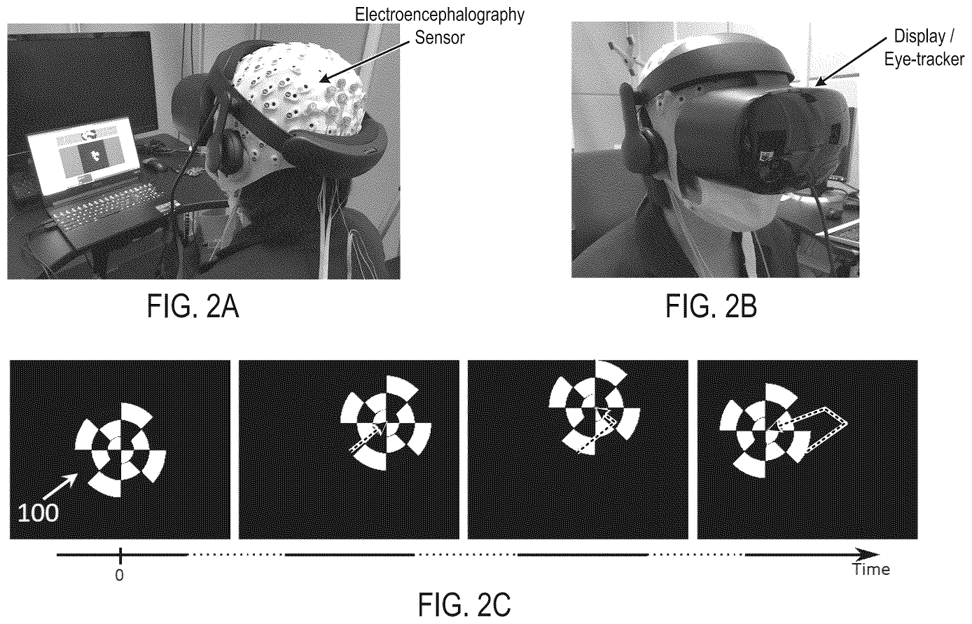

FIG. 2A shows a system that is used for an experiment according to an example embodiment. In some implementations, the system includes an EEG device. FIG. 2B shows another view of the system shown in FIG. 2A to illustrate a virtual reality (VR) head-mounted display (HMD) part of the system placed on top of an EEG cap. FIG. 2C shows an example sequence of movements of a visual flicker 100, according to an example embodiment.

The new portable BCI platform can employ a unique, moving visual flicker to capture both (a) delays in afferent signal conduction to the occipital cortex and (b) efferent oculomotor dysfunction. Participants will fix their gaze at the center of the stimulus so that it would simultaneously elicit VEPs and saccadic eye movements. The amplitude of VEPs and saccadic eye movements will be quantified by electroencephalography (EEG) and electromyography (EOG)/an eye-tracker, respectively, to assess afferent and efferent visual dysfunction.

During an experimental data stage, mfSSVEP and EOG data can be collected from 10 MS and 10 control participants (subjects) while performing the moving visual flicker pursuit task.

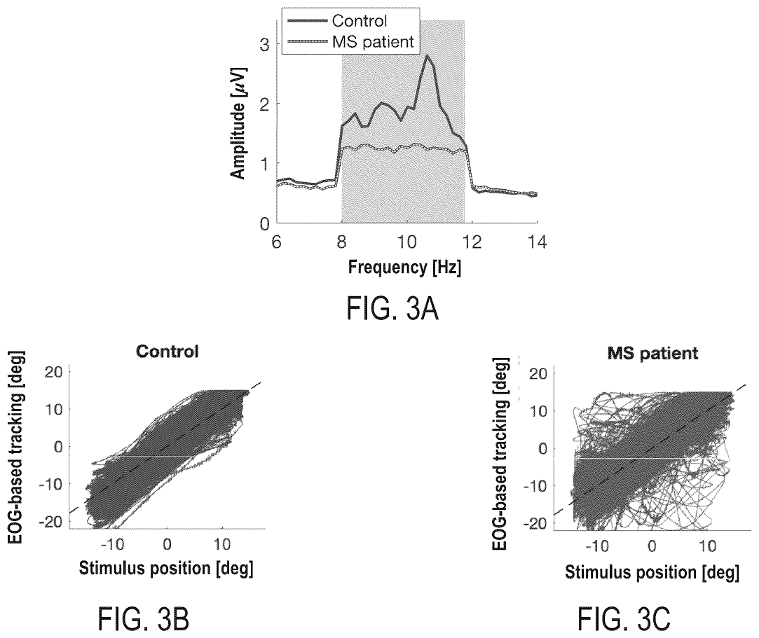

FIG. 3A shows example amplitude spectra of mfSSVEPs. FIG. 3B shows example EOG-based eye tracking data for a control subject and an MS patient.

The results show in FIGS. 3A and 3B indicate that lower mfSSVEP amplitude and poorer tracking performance is obtained in the MS patient than the healthy control.

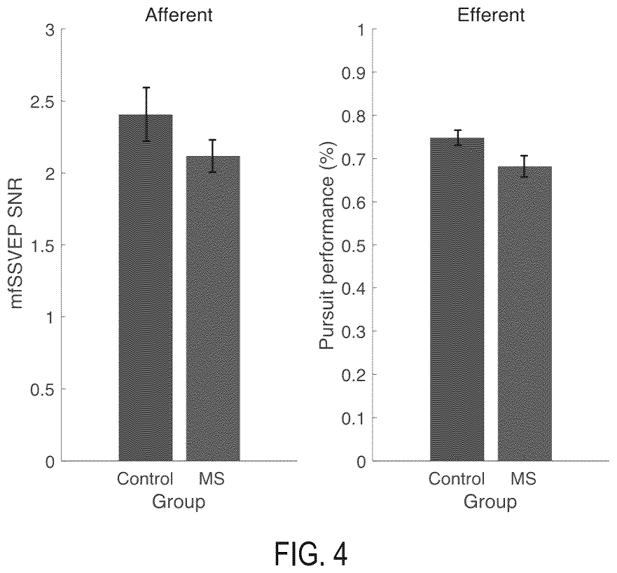

FIG. 4 shows averaged mfSSVEP amplitude (Left) and the performance of pursuit task (Right) in the groups of healthy controls and MS patients. MS participants showed decreased SNR of mfSSVEPs and eye-tracking performance.

A mobile multi-focal steady-state VEP (mfSSVEP)-based brain-computer interface (BCI) platform with an embedded eye-tracking component according to the disclosed technology can capture simultaneous data on afferent and efferent visual function. These are rarely measured together and thus general understanding of the degree to which dysfunction correlates in the two domains and influences respective outcome measures is limited. Disorders of fixation can impact apparent visual acuity and, conversely, the ability to detect visual stimuli may impair eye-tracking Many neuro-ophthalmologist researchers specialize in either afferent or efferent research, not both, as equipment for both can be expensive and time-consuming to manage. The BCI platform according to the technology disclosed herein can greatly facilitate research on the correlations between the afferent and efferent measures and support the development of novel indices of MS-related visual dysfunction.

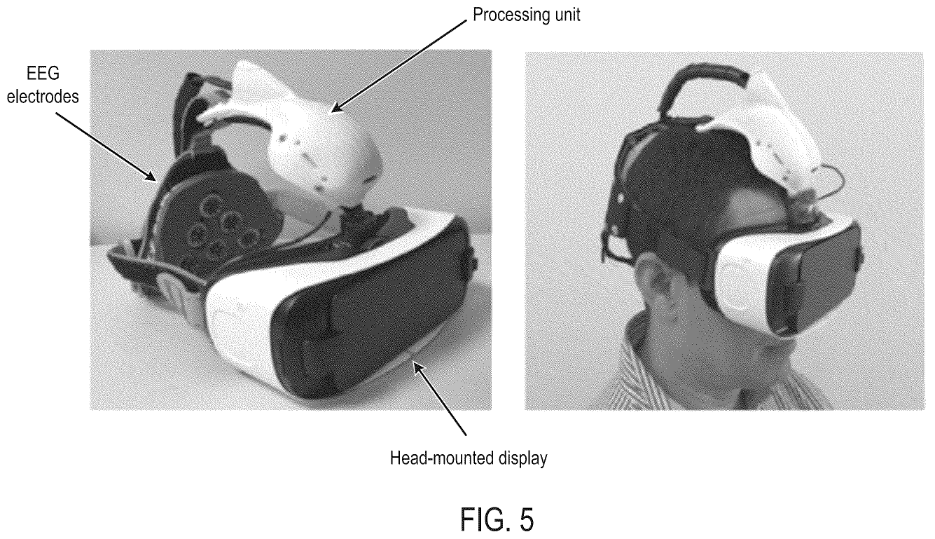

FIG. 5 shows a device implemented based on an example embodiment. The device is a portable device which includes wireless, no-prep EEG electrodes, a processing unit (which includes, e.g., one or more processors and a memory), and a head-mounted display.

Visual evoked potential (VEP) techniques are commonly used to diagnose demyelination injury of the optic nerve. However, these methods require non-portable devices in the clinic-or laboratory-based settings, requiring cumbersome setup and placement of traditional electrodes. Data collection requires skin preparation and gel application to ensure good electrical conductivity between sensors and skin, which are time-consuming and uncomfortable for the patients. To address this issue, a device implemented based on an example embodiment of the disclosed technology includes wireless, easy-to-wear EEG and electrooculogram (EOG) systems and a virtual-reality (VR) head-mounted display (HMD). The device uses a VR HMD with a smartphone for stimulus presentation (FIG. 5). The device uses customized flexible polymer-based EEG electrodes and foam-based EOG electrodes for no-preparation wearing. The miniature and wireless EEG amplifier (e.g., 3 cm×4.5 cm×1.5 cm in size) can sample up to 16 EEG/EOG channels synchronously at 500 sps. The Bluetooth communications among the VR goggle, the EEG amplifier, and the console (tablet computer or smartphone, for example) allow the system to be used completely untethered anytime anywhere. The Wi-Fi-Internet also supports wireless data transfer.

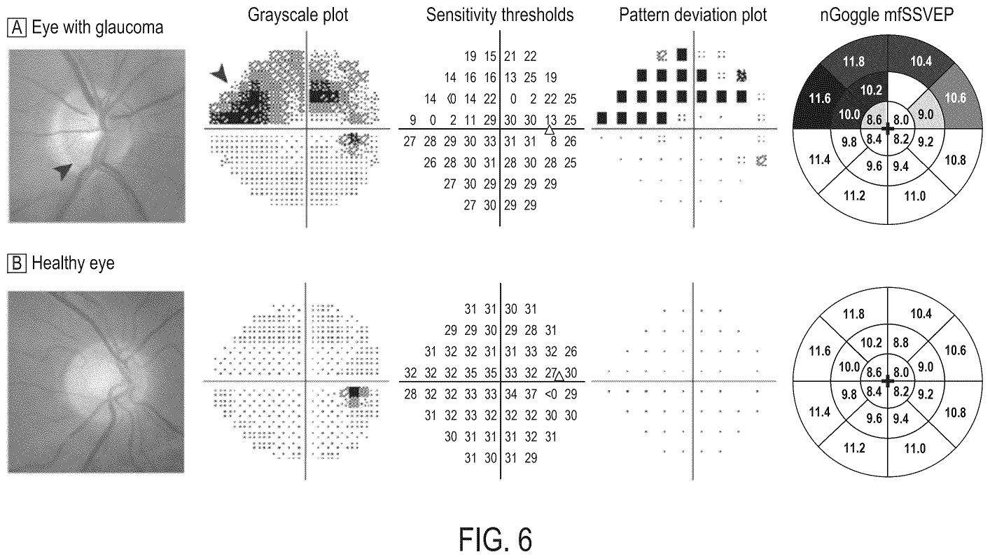

FIG. 6 shows some example results of visual field assessments from nGoggle mfSSVEP and standard perimetry of glaucomatous and healthy eyes.

Referring to FIG. 6, the device shown in FIG. 5 (referred to as nGoggle) can be used, for example, to assess optic nerve injury in glaucoma patients. In addition to the conventional transient full-field VEP techniques, the device can use a multifocal steady-state visual-evoked potential (mfSSVEP) method toward a faster assessment and better signal-to-noise ratio (SNR). To test the ability of diagnosing glaucomatous visual deficits, tests involving 62 eyes from 33 participants with glaucoma and 30 eyes from 17 healthy controls were performed. In this application of the nGoggle, the visual field locations of deficits in the mfSSVEP signals are used as a proxy for visual field defects. The results showed that the nGoggle mfSSVEP metrics are able to distinguish glaucomatous from healthy eyes with area under the receiver operating characteristic curve (AUROC) of 0.92 (95% CI: 0.86-0.96), which is significantly higher than that of 0.81 (95% CI: 0.72-0.90) for standard automated perimetry (SAP) (p=0.046) (FIG. 6). In addition, eyes with early-stage preperimetric glaucoma (PPG), which has evidence of glaucomatous optic neuropathy on optic disc stereophotographs but normal visual field results on SAP, may also be tested using nGoggle.

In some example embodiments, the device (e.g., nGoggle) implemented based on some embodiments of the disclosed technology can include an IR-camera-based eye tracker to capture fixation losses and MS oculomotor dysfunction.

An example 3D eye-tracking method implemented based on some embodiments of the disclosed technology uses electromyography (EOG) signals. In an experiment to test the method, participants are instructed to continuously gaze at a moving target in 3-D space. From each of 10 subjects, 8-channel EOG signals are acquired, according to the method, while the subjects are performing the experimental task. The gaze direction and fixation depth are estimated, according to the method, by analyzing the collected EOG signals using a linear regression analysis and a nonlinear regression based on a neural network (NN). The linear analysis showed averaged azimuth (horizontal) and elevation (vertical) angle errors of 2.9±2.1 and 4.4±3.3 degrees, respectively, and averaged fixation distance errors of 13.11% (7.5±5.6 cm). The nonlinear NN-based method showed averaged errors for azimuth and elevation angle of 2.3±1.6 and 3.2±2.6 degrees, respectively, and average fixation distance error of 10.3±10.0% (5.7±4.7 cm). The method uses EOG signals as a basis for eye tracking in 3-D space.

Devices implemented based on some embodiments of the disclosed technology include VR HMD with integrated sensors for the purpose of collecting synchronous physiological data from subjects experiencing the VR-delivered stimuli. Some example embodiments include an open source virtual reality (OSVR) HDK 2 goggle hardware that allows implementing VR environments of custom designs. An example embodiment includes an eye-tracking system, electrodes for hair, and electrodes for skin. In an example embodiment, EEG and EOG signals are acquired using a miniature amplifier on a PCB that supports up to 16 analog differential input channels and 1 external reference, sampling at 250 or 500 Hz, with a signal passband of 1-100 Hz.

Devices implemented based on some embodiments of the disclosed technology collect physiological data while subjects perceive repetitive visual stimulation presented on the VR display of the device. Two types of visual stimuli can be utilized: 1) a full-field stimulus, and 2) a multi-focal stimulus. The full-field stimulus includes, e.g., a circle covering the central 25° of the visual field that is modulated by a single frequency at, e.g., 10 Hz to induce conventional SSVEP signals. In contrast, the multi-focal stimulus allows many areas of the retina to be stimulated simultaneously and separate responses from each part of the visual field to be obtained. A layout of the multi-focal stimulus includes a plurality of sectors. For example, a layout of the multi-focal stimulus includes 20 sectors in three concentric rings (subtending, e.g., 6°, 15°, and 25° of the visual field). All sectors flicker concurrently at different frequencies with a certain resolution. For example, all sectors flicker concurrently at different frequencies ranging from 8 to 11.8 Hz with a frequency resolution of 0.2 Hz. The frequency range and spacing can be flexible. In some implementations, the stimulus layout and the frequency assignment in a multi-focal stimulus can be flexibly designed according to a target application. Subjects can be asked to maintain fixation at the center of the visual stimuli throughout the entire experiment. The subjects' fixation can be monitored by the eye-tracking system and EOG sensor(s) of the device in real-time.

In some example embodiments, the device can include EOG sensors or eye-tracking system which can be used to assess fixation losses and capture abnormalities in saccades and smooth pursuit eye movements. MS patients have slowed saccadic movements (increased latencies, decreased velocities) and abnormal smooth pursuit including increased saccadic intrusions. Devices and methods according to the technology disclosed herein can allow objectively assessing the eye-movement behaviors while subjects are fixating their vision on a moving target presented on the HMD of the device. The fixation target can move in 2-D space rendered on the screen of the HMD randomly and/or smoothly. Stimuli according to some example embodiments can allow simultaneously assessing afferent and efferent visual pathway injury. Such stimuli can be produced using a stationary multi-focal repetitive visual stimulation and a moving fixation cross separately presented or using a moving multi-focal repetitive visual stimulation toward fast diagnosis.

The following variables can be used by methods according to some example embodiments: demographic variables (age, sex, race, education level); key clinical variables including disease subtype, history of optic neuritis, disease duration, current relevant medications, recent clinical MRI findings (which can be extracted from reports), bedside exam findings (including visual acuity and oculomotor deficits if present); a battery of MS clinical outcome measures collected at routine visits (e.g., EDSS, timed 25-foot walk); a visual quality of life questionnaire validated for MS patients (NEI-VFQ-25 with 10 item neuro-ophthalmologic supplement); optical coherence tomography (OCT) measurements including peripapillary retinal nerve fiber layer thickness (RNFL) and macular metrics (both correlated with MS-related optic nerve injury); and/or full-field VEPs (which can be collected at, e.g., a neurophysiology lab).

An example method implemented based on some embodiments of the disclosed technology includes displaying, for a user (also referred to as a subject or person or participant in this patent document), a VR task (e.g., a series of VR images on a head-mounted display of a system according to an example embodiment) while evoked potential(s) and eye-tracking data of the user are recorded using an EEG sensor and an eye-tracker or the system. Methods according to the disclosed technology can be performed in, e.g., routine exam rooms and can include repeating measurements or data collection steps a number of times in order to obtain intra-class correlation coefficients. Real-time information (e.g., data or feedback) can be provided to a health care provider or practitioner through a reporting functionality (e.g., a data transfer channel) to, e.g., identify excessive fixation losses impairing data use. Qualitative comments from the users can be collected using, e.g., a questionnaire.

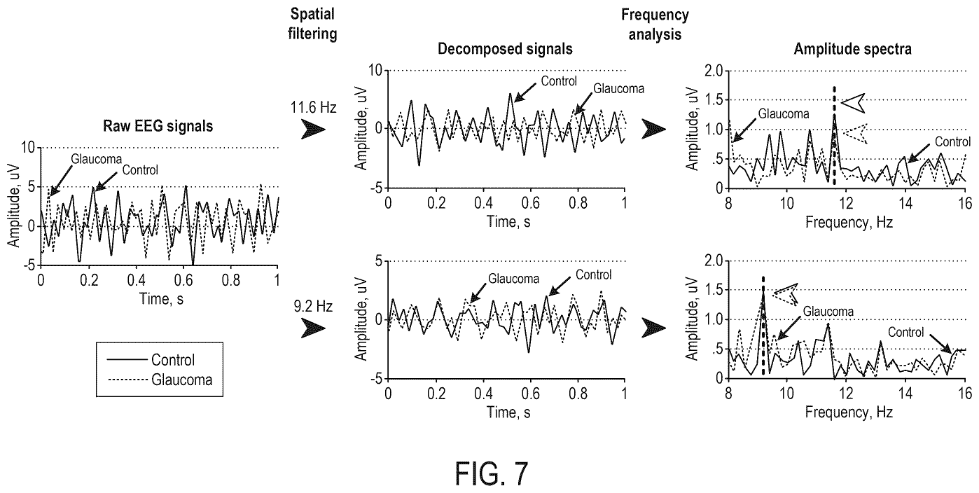

FIG. 7 shows a signal processing pipeline that can be used to quantify mfSSVEP signals based on some embodiments of the disclosed technology. A spatial-filtering method according to an example embodiment can decompose a raw EEG signal into multiple frequency components.

In order to obtain information related to the afferent visual pathway in the users, methods and devices according to the disclosed technology can use signal-processing techniques such as fast Fourier transform (FFT) and/or canonical correlation analysis (CCA) to perform feature extraction and spectral analysis of the EEG data collected from the wearable EEG sensors of the devices. Furthermore, methods implemented based on some embodiments of the disclosed technology can include mitigating artifacts contaminating EEG signals using, e.g., a task-related component analysis (TRCA)-based spatial filtering technique. The TRCA-based spatial filtering technique can be also used for enhancing the classification accuracy of frequency-tagged SSVEPs. This spatial filtering technique can also be used to decompose the mfSSVEP signals into each frequency component (FIG. 7). In some implementations, these techniques can provide separate frequency features corresponding to each sector in the multi-focal stimulus. Signal-cleaning methods based on the techniques mentioned above can be applied to the collected mfSSVEP data, followed by extracting frequency features in mfSSVEPs via, e.g., the FFT and/or CCA. The full-field SSVEP signals can also be quantified using the same approach.

Methods and systems implemented based on some embodiments of the disclosed technology also allow investigating the efferent visual pathway in the participants/users. For example, they can allow quantifying saccadic eye movements using the EOG data collected by the HMD EOG sensors. Methods according to some example embodiments include removing baseline drifts (e.g., the ones that are unrelated to eye movements whose possible sources are, e.g., interference background signals or electrode polarization). Methods according to certain example embodiments include using a trained linear regression and feed-forward neural network (NN) model. The model can be trained and/or used with the artifact-free EOG signals and can be used for tracking subjects' smooth pursuit.

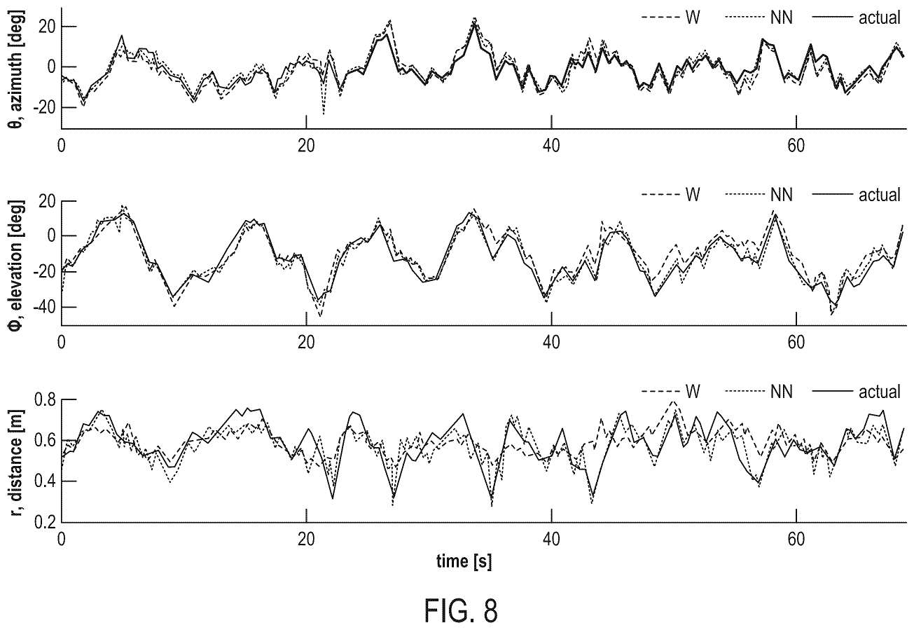

FIG. 8 shows example traces of angular position in a smooth pursuit experiment, showing the estimated angular positions from EOG-based and camera-based eye tracking. In some example embodiments, a continuous wavelet transform (CWT) with, e.g., a Haar mother wavelet can be applied to baseline-corrected EOG signals to detect the onset of saccadic eye movements. According to some example embodiments, the amplitude and latency of saccadic eye movements can be quantified as feature values to differentiate diseased from healthy eyes.

Methods implemented based on some embodiments of the disclosed technology can use different descriptive statistics measures, including means, medians, and frequencies, as appropriate. All variables used by the methods can be assessed for the assumption of normal distribution and data transformations applied before regression analysis if needed. Interclass Correlation Coefficients (ICCs) can be generated for any metrics to determine test re-test reliability. Each eye can be a unit of observation. Generalized estimating equations (GEE) models can be used and inter-eye correlations can be adjusted for within the same participant. Potential confounders which can be evaluated according to the disclosed methods include age, sex, MS medication use, and disease duration. Models can be determined by backward selection.

Wearable devices and systems implemented based on some embodiments of the disclosed technology can be used to detect demyelination-related afferent visual pathway injury in the eyes of MS patients. Methods according to some example embodiments include comparing the nGoggle mfSSVEP and/or full-field SSVEP signals for a) multiple sclerosis (MS) optic neuritis (ON) eyes vs. healthy controls, b) MS ON eyes vs. MS non-ON eyes, and MS non-ON eyes vs. healthy controls. Increased latencies and decreased amplitudes in the evoked potential signals between MS ON eyes and healthy controls can be expected and non-ON MS eyes can be expected to have intermediate values between the MS ON and healthy control eyes. Methods according to some embodiments of the disclosed technology can use unadjusted and adjusted regression analyses as well as employ AUROC (area under the receiver operating characteristic curve) analyses to generate estimates of their sensitivity and specificity. Results obtained using nGoggle mfSSVEP and full field SSVEP can be related to (e.g., correlated with) those obtained using the current standard of care full-field VEP (for example, Spearman's correlation coefficient can be used). Methods according to the disclosed technology can use multivariable GEE models to determine the association of the metrics obtained using nGoggle mfSSVEP and full field SSVEP with standard VEP P100 latencies. Adjustment for and stratified analyses can be performed based on eye classification: MS ON eye, MS non-ON eye and healthy eye. The nGoggle mfSSVEP and full field SSVEP signals can be related to (e.g., correlated with) structural evidence of RGC injury obtained by optical coherence tomography. Evoked potential abnormalities can be expected to associate with peripapillary retinal nerve fiber loss and decreased macular volumes. GEE models adjusted for relevant confounders can be used by any of the methods according to the disclosed technology.

In some example embodiments, concurrently collected eye-tracker signals are employed to evaluate oculomotor function. Persons with MS can be expected to have greater fixation losses and impaired smooth and saccadic eye movements, compared to healthy controls.

Methods implemented based on some embodiments of the disclosed technology can relate (e.g., correlate) efferent abnormalities with the patient's overall disability level (expanded disability status scale score, EDSS). As there are typically two eyes but only one EDSS per patient, some example embodiments can use the eye with the worst performance scores.

Relative performance and associations between afferent and efferent measurements can be established by methods implemented based on some embodiments of the disclosed technology. Relative performance of different metrics in distinguishing MS eyes from controls and determining the level of disease severity within MS participants can be established. For that purpose, regression and AUROC analyses can be used to determine the relative strengths of associations of the nGoggle metrics with case status and the sensitivity and specificity of the nGoggle-generated data to detect pathology.

Methods implemented based on some embodiments of the disclosed technology can also use an association of performance on the mfSSVEP with the EOG measures. With an unprecedented ability to track these (mfSSVEP and EOG) measures concurrently provided by methods and devices disclosed herein, afferent and efferent visual function interdependence in participants with MS can be determined using, e.g., Spearman correlation coefficients and regression analysis adjusting for the above-mentioned possible confounders. Analyses can also be stratified by history of optic neuritis.

Devices and systems disclosed herein could be used, for example, in neurologist or ophthalmologist office, emergency room or inpatient hospitals as well as in large international clinical trials of neurological diseases like MS. Additional applications include highly prevalent diseases of brain trauma/concussion, Alzheimer's disease and Parkinson's.

The ability to deploy a wearable device to capture afferent and efferent visual dysfunction provided by the disclosed technology will enable i) expediting diagnosis and treatment, ii) enhancing the care of MS patients outside tertiary referral centers, and iii) facilitating more rapid development of remyelinating and neuroprotective agents that can be screened cost-effectively with quantitative, multi-domain visual outcome measures.

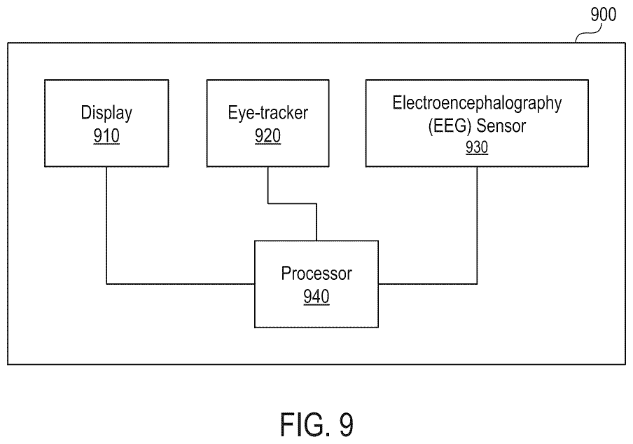

FIG. 9 shows an example of a wearable system for assessing afferent and efferent visual functions based on some embodiments of the disclosed technology.

In some implementations, a system 900 may include a display 910 configured to be placed in front of a face of a user and provide visual stimuli to the user to elicit eye movements, an eye-tracker 920 configured to be placed on a head of the user to measure electrical activity in a brain of the user that occurs in response to the visual stimuli, an electroencephalography (EEG) sensor 930 configured to be placed on a head of the user to measure electrical activity in a brain of the user that occurs in response to the visual stimuli; an eye-tracker configured to track the eye movements, and a processor 940 coupled to the display 910, the eye-tracker 920, and the EEG sensor 930 to: cause the visual stimuli to be presented on the display 910; obtain an electroencephalography signal from the EEG sensor 930; obtain eye-tracking measurements from the eye-tracker 920; and determine, based on the electroencephalography signal and the eye-tracking measurements, information associated with the afferent and efferent visual functions.

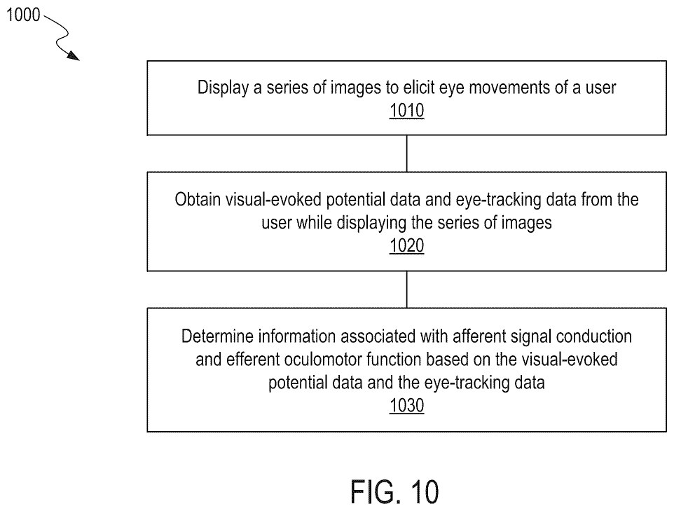

FIG. 10 shows an example method of quantifying multiple sclerosis related visual dysfunctions based on some embodiments of the disclosed technology.

In some implementations, a method 1000 includes, at 1010, displaying a series of images to elicit eye movements of a user, at 1020, obtaining visual-evoked potential data and eye-tracking data from the user while displaying the series of images, and at 1030, determining information associated with afferent signal conduction and efferent oculomotor function based on the visual-evoked potential data and the eye-tracking data.

Therefore, various implementations of features of the disclosed technology can be made based on the above disclosure, including the examples listed below.

1. A wearable system for assessing afferent and efferent visual functions, comprising: a display configured to be placed in front of a face of a user and provide visual stimuli to the user to elicit eye movements; an electroencephalography (EEG) sensor configured to be placed on a head of the user to measure electrical activity in a brain of the user that occurs in response to the visual stimuli; an eye-tracker configured to track the eye movements; and a processor coupled to the display, the electroencephalography sensor, and the eye-tracker to: cause the visual stimuli to be presented on the display; obtain an electroencephalography signal from the EEG sensor; obtain eye-tracking measurements from the eye-tracker; and determine, based on the electroencephalography signal and the eye-tracking measurements, information associated with the afferent and efferent visual functions.

2. The system of example 1, wherein the eye-tracker includes an electromyography (EOG) sensor.

3. The system of example 2, wherein the EOG sensor includes a plurality of signal collection channels.

4. The system of example 2, wherein the EOG sensor includes foam-based EOG electrodes.

5. The system of example 1, wherein the eye-tracker includes an infrared (IR) video camera configured to capture fixation losses and multiple sclerosis (MS) oculomotor dysfunction. For example, the eye-tracker includes an infrared (IR) video camera configured to generate eye-tracker signals that are used to capture fixation losses and multiple sclerosis (MS) oculomotor dysfunction.

6. The system of example 1, wherein the eye-tracker is configured to capture gaze direction or fixation depth. For example, the eye-tracker is configured to generate eye-tracker signals that are used to capture gaze direction or fixation depth

7. The system of example 6, wherein the gaze direction or fixation depth is determined using a linear regression analysis or a nonlinear regression based on a neural network (NN).

8. The system of example 1, wherein the EEG sensor is configured to produce multi-focal steady-state visual evoked potential (mfSSVEP) measurements.

9. The system of example 1, wherein the information associated with the afferent and efferent visual functions includes information associated with afferent signal conduction to an occipital cortex and information associated with efferent oculomotor function.

10. The system of example 9, wherein the information associated with afferent signal conduction to the occipital cortex includes information associated with delays in afferent signal conduction to the occipital cortex.

11. The system of example 1, wherein the EEG sensor includes flexible polymer-based EEG electrodes.

12. The system of example 1, wherein the display includes a head-mounted display (HMD).

13. The system of example 1, wherein the visual stimuli include at least one of a full-field stimulus or a multi-focal stimulus.

14. The system of example 1, wherein the visual stimuli include a moving visual flicker.

15. The system of example 14, wherein the information associated with the afferent and efferent visual functions includes at least one of: delays in afferent signal conduction to the occipital cortex; or efferent oculomotor dysfunction.

16. A method of quantifying multiple sclerosis related visual dysfunctions, comprising: displaying a series of images to elicit eye movements of a user; obtaining visual-evoked potential data and eye-tracking data from the user while displaying the series of images; and determining information associated with afferent signal conduction and efferent oculomotor function based on the visual-evoked potential data and the eye-tracking data.

17. The method of example 16, further comprising: comparing the visual-evoked potential data and the eye-tracking data to reference visual-evoked potential data and reference eye-tracking data corresponding to healthy eyes to determine visual dysfunctions.

18. The method of example 17, wherein the comparing of the visual-evoked potential data includes determining increased latencies and decreased amplitudes in the visual-evoked potential data between MS ON eyes and healthy controls.

19. The method of example 16, wherein the visual-evoked potential data includes at least one of a multi-focal steady-state visual evoked potential (mfSSVEP) or full-field steady-state visual evoked potential (SSVEP).

20. The method of example 16, wherein the eye-tracking data includes electromyography (EOG) data.

21. The method of example 20, wherein the visual-evoked potential data is determined by the EEG data to assess afferent and efferent visual dysfunction.

22. The method of example 16, wherein the visual-evoked potential data includes electroencephalography (EEG) data.

23. The method of example 22, wherein the eye movements are determined by the EOG data to assess afferent and efferent visual dysfunction.

24. The method of example 16, wherein the series of images include moving visual flickers.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

It is understood that the various disclosed embodiments may be implemented individually, or collectively, in devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Only a few implementations and examples are described and other implementations, enhancements and variations can be made based on what is described and illustrated in this patent document.

1. A wearable system for assessing afferent and efferent visual functions, comprising:
a display configured to be placed in front of a face of a user and provide visual stimuli to the user to elicit eye movements;
an electroencephalography (EEG) sensor configured to be placed on a head of the user to measure electrical activity in a brain of the user that occurs in response to the visual stimuli;
an eye-tracker configured to track the eye movements; and
a processor coupled to the display, the electroencephalography sensor, and the eye-tracker to: cause the visual stimuli to be presented on the display; obtain an electroencephalography signal from the EEG sensor; obtain eye-tracking measurements from the eye-tracker; and determine, based on the electroencephalography signal and the eye-tracking measurements, information associated with the afferent and efferent visual functions.

2. The system of claim 1, wherein the eye-tracker includes an electromyography (EOG) sensor.

3. The system of claim 2, wherein the EOG sensor includes a plurality of signal collection channels.

4. The system of claim 2, wherein the EOG sensor includes foam-based EOG electrodes.

5. The system of claim 1, wherein the eye-tracker includes an infrared (IR) video camera configured to generate eye-tracker signals that are used to capture fixation losses and multiple sclerosis (MS) oculomotor dysfunction.

6. The system of claim 1, wherein the eye-tracker is configured to generate eye-tracker signals that are used to capture gaze direction or fixation depth.

7. (canceled)

8. The system of claim 1, wherein the EEG sensor is configured to produce multi-focal steady-state visual evoked potential (mfSSVEP) measurements.

9. The system of claim 1, wherein the information associated with the afferent and efferent visual functions includes information associated with afferent signal conduction to an occipital cortex and information associated with efferent oculomotor function.

10. The system of claim 9, wherein the information associated with afferent signal conduction to the occipital cortex includes information associated with delays in afferent signal conduction to the occipital cortex.

11. The system of claim 1, wherein the EEG sensor includes flexible polymer-based EEG electrodes.

12. The system of claim 1, wherein the display includes a head-mounted display (HMD).

13. The system of claim 1, wherein the visual stimuli include at least one of a full-field stimulus or a multi-focal stimulus.

14. The system of claim 1, wherein the visual stimuli include a moving visual flicker.

15. The system of claim 14, wherein the information associated with the afferent and efferent visual functions includes at least one of: delays in afferent signal conduction to the occipital cortex; or efferent oculomotor dysfunction.

16. A method of quantifying multiple sclerosis related visual dysfunctions, comprising:
displaying a series of images to elicit eye movements of a user;
obtaining visual-evoked potential data and eye-tracking data from the user while displaying the series of images; and
determining information associated with afferent signal conduction and efferent oculomotor function based on the visual-evoked potential data and the eye-tracking data.

17. The method of claim 16, further comprising:
comparing the visual-evoked potential data and the eye-tracking data to reference visual-evoked potential data and reference eye-tracking data corresponding to healthy eyes to determine visual dysfunctions.

18. (canceled)

19. The method of claim 16, wherein the visual-evoked potential data includes at least one of a multi-focal steady-state visual evoked potential (mfSSVEP) or full-field steady-state visual evoked potential (SSVEP).

20. The method of claim 16, wherein the eye-tracking data includes electromyography (EOG) data.

21. (canceled)

22. The method of claim 16, wherein the visual-evoked potential data includes electroencephalography (EEG) data.

23. (canceled)

24. The method of claim 16, wherein the series of images include moving visual flickers.
